(19) United States
(12) Patent Application Publication
(10) Pub. No.: US 2003/0229346 A1
(43) Pub. Date: Dec. 11, 2003

(54) Screw for fixing atlantoaxial joint

(75) Inventors: Kazuya Oribe, Tokyo (JP); Hiroshi Takamido, Nagoya-shi (JP)

Correspondence Address: GREENBLUM & BERNSTEIN, P.L.C., 1950 ROLAND CLARKE PLACE, RESTON, VA, 20191, US

(73) Assignee: Showa Ika Kohgyo Co., Ltd., Aichi (JP)

(21) Appl. No.: 10/431,474

(22) Filed: May 8, 2003

(30) Foreign Application Priority Data
May 9, 2002 (JP) JP2002-134261

(51) Int. Cl. A61B 17/70; A61B 17/86

(52) U.S. Cl. 606/61; 606/73

(57) ABSTRACT

A construction, wherein a cutter portion for attachment 13 is provided at one end of a screw 12 having a through-hole 17 in the shaft center, a cutting edge part for attachment 13 which acutely forms a pair of facing protrusions 20 formed by a pair of inclined planes 21, and protrusions 20 which have cutter blades 13A.

### BACKGROUND OF THE INVENTION

1. Field of the Invention

The present invention relates to screws for fixing atlantoaxial joints used for spondylodesis, a medical treatment for spondylous patients such as atlantoaxial joint instability.

2. Description of the Related Art

As shown in FIGS. 1A to 1D, as an example of a method for fixing an atlantoaxial joint of an atlanto (the first cervical vertebra) and an axis (the second cervical vertebra), there is a method in which a pair of right and left screws 5 are screwed and embedded into lateral masses 4 of an atlanto 1 from inferior articular processes 3 of an axis 2.

More specifically, after stomas 22 are bored in advance at the positions where the screws 5 are to be screwed and embedded into the first and the second cervical vertebrae to the axis 2 and the atlanto 1, the screws 5 are screwed and embedded in the direction of R₁ in FIG. 1B. Ends 7 of the screws 5 used in this example have flat shapes, i.e. shapes which are cut close to perpendicular to a longitudinal direction of the screws 5. Therefore, an attachment of the screws 5 to the atlanto 1 and the axis 2 is bad, and particularly when the screws 5 are screwed into a joint 8 where the atlanto 1 and the axis 2 are lapped over, there has been problems such as that the screws 5 are not screwed into articular surface stomas 9 of the atlanto 1, the end faces in the traveling direction of the screws 5 abut the atlanto 1, the atlanto 1 is pushed away from the axis 2 by component force F₂ of pressing force F₁ of the screws 5 (FIG. 1B).

Therefore, when the screws S are screwed and fixed, actions has been taken in such a way that, after formation of embedding parts 10 where the ends of the screws 5 are slightly embedded in the articular surface stomas 9 of the atlanto 1 is confirmed, the screws 5 are temporarily drawn back in the direction of an arrow R₂ in FIG. 1C. Thereafter, when an upper part of the atlanto 1 is returned to the original position by its own weight (FIG. 1C), the screws 5 are threaded again.

In the above method, the screws 5 should be pulled back and inserted, resulting in bad operating efficacy.

### SUMMARY OF THE INVENTION

The invention is devised in consideration of the conventional problems as described above. According to the present invention, there is provided a screw for fixing an atlantoaxial comprising, a shaft body having a through-hole in a center of the shaft body from one end to the other end, a screw part provided on one end of the shaft body, and a cutting portion for screwing is provided on the one tip end of the screw part.

### BRIEF DESCRIPTION OF THE DRAWINGS

FIGS. 1A to 1D are explanatory drawings illustrating a conventional example of a method for fixing atlantoaxial joints.

FIGS. 2A to 2C are explanatory drawings illustrating a usage example of screws for fixing atlantoaxial joints according to the invention.

FIGS. 3A to 3C are explanatory drawings for the screws for fixing atlantoaxial joints according to the invention.

### DETAILED DESCRIPTION OF THE INVENTION

An embodiment of the present invention will be described below based on accompanying drawings.

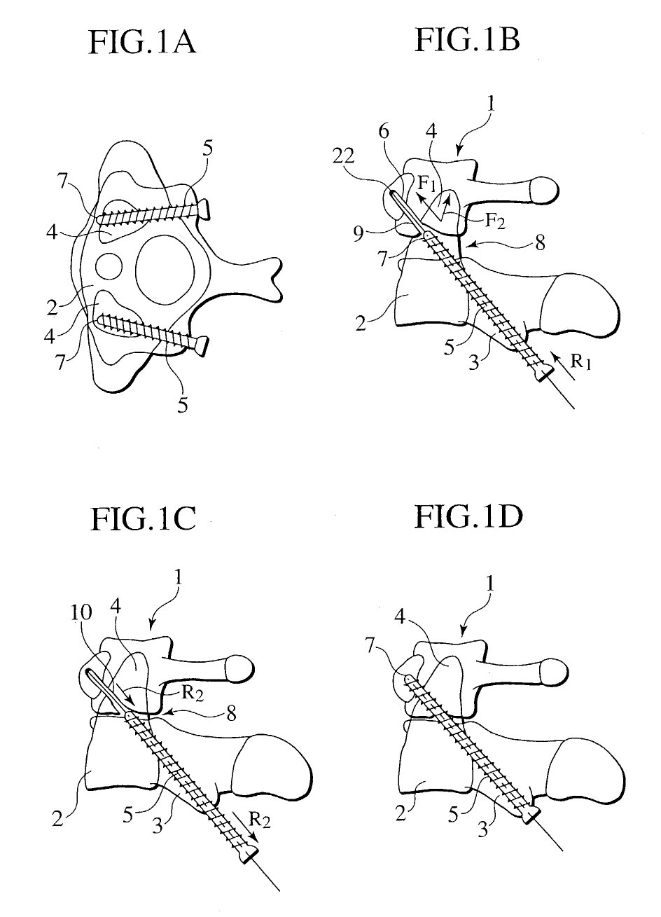

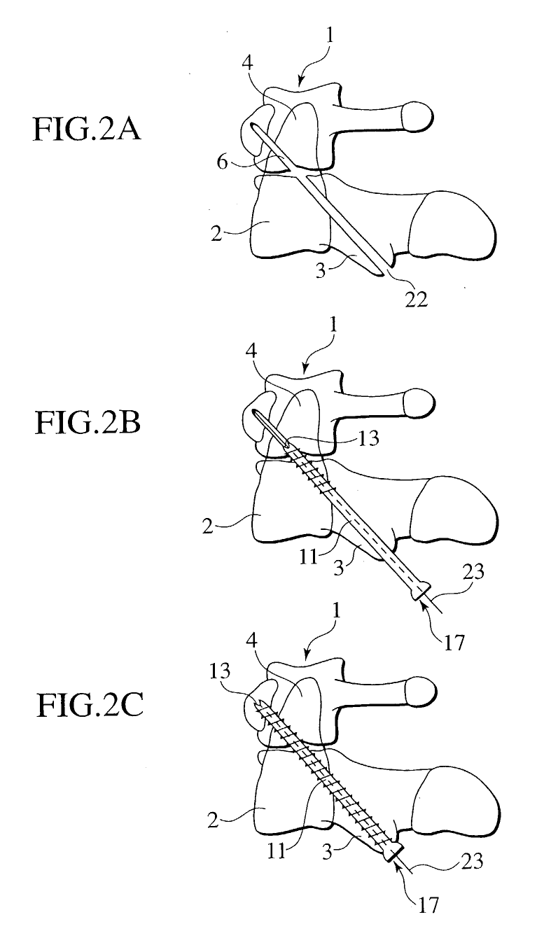

As shown in FIGS. 2A to 2C, a screw for fixing operation of an atlantoaxial joint 11 (hereinafter referred to as “screw 11”) used for the fusion of an atlantoaxial joint of an atlanto 1 and an axial 2 is diagonally screwed and embedded into a lateral mass 4 of the atlanto 1 from an inferior articular process 3 of the axis 2 as the conventional screws 5 (refer to FIG. 1A).

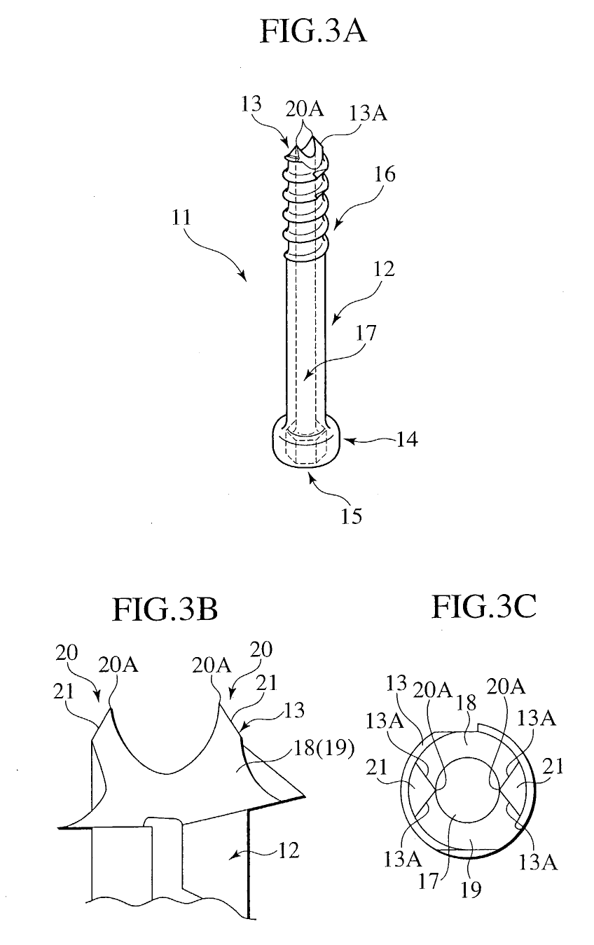

As shown in FIG. 3A, the screw 11 has a cutter portion 13 provided on one end side of a shaft body 12, and a head with a large diameter 14 having a tool engaging hole 15, which is provided on the other end side of the shaft body 12.

On one end side of the shaft body 12, a screw 16 is provided with the cutter portion 13. In the center of the shaft body 12, a through-hole 17 is provided from the head 14 to the cutter portion 13.

As shown in FIGS. 3B and 3C, the cutter portion 13 has a pair of facing protrusions 20 provided by forming the first inclined plane 18 and the second inclined plane 19 in approximately symmetric figure at the end of the shaft body 12. The clearance between inclined planes 18, 19 is getting small from the shaft body 12 to a tip portion thereof. Furthermore, on the outer peripheral surface on the end side of the protrusions 20, taper faces 21 whose end sides are oriented inside are formed to acutely form ends 20A of the protrusions 20.

In other words, cutter blades 13A are formed at crossing portions of the inclined planes 18, 19 and the taper faces 21.

A method of fixing cervical vertebrae by using the screw 11 is described with reference to FIGS. 2A to 2C as below:

In order to link and fix the axis 2 and the lateral mass 4 by the screw 11, firstly, a stoma 22 is opened diagonally to the cervical vertebrae by a boring tool such as an air drill and the like from the inferior articular process 3 of the axis 2 to the lateral mass 4 of the atlanto 1, and secondly, a guiding line 23 such as a wire (not shown), a guide pin (not shown) and the like is inserted in the through-hole 17 of the screw 11, and then the screw 11 is screwed into the stoma 22 by using the guiding line 23 as a guide. In other words, the guiding line 23 is used as a guide by inserting it through the through-hole 17 of the screw 11.

When the screw 11 is screwed into a stoma 6 which is bored in the lateral mass 4 of the atlanto 1 from the inferior articular process 3 of the axis 2, as the screw 11 is pressured and fixed along with the rotation thereof, the cutter blades 13A of the cutting portion 13 are screwed toward inside over the scraping inside of the stoma 22.

As the ends 20A of the both protrusions 20 can be elastically deformed so that they can close each other due to component forces which act inward on the cutter blades 13A in cutting, screwing into the stoma 22 becomes easy.

According to the embodiment, at the start of the screwing, an end diameter of the screw 11 in the direction of screwing becomes small due to the component forces, so that screwing into the target is easy to perform. In the course of screwing, the screw 16 is surely embedded in the vertebrae due to reaction force which makes the diameter return to the original length against the component forces. By further continuing to screw the screw 16, the atlantoaxial joint can be easily fixed.

Additionally, a trend that the screw 11 pushes away the atlanto 1 in screwing can be restrained, so that the conventional problem as mentioned above can be solved.

Therefore, as the atlanto 1 is never spaced from the axis 2 by the screw 11, there is no need to take conventional actions such as that the screw 5 embedded in a embedding part 10 of the atlanto 1 is temporarily backed up, and the screw 5 is screwed again after the atlanto 1 returns to the original position, so that efficiency can be improved.

The entire content of a Japanese Application No. P2002-134261 with a filing date of May 9, 2002 is herein incorporated by reference.

Although the invention has been described above by reference to certain embodiments of the present invention, the invention is not limited to the embodiments described above and will occur to those skilled in the art, in light of the teachings. The scope of the invention is defined with reference to the following claims.

What is claimed is:

1. A screw for fixing an atlantoaxial comprising:
a shaft body having a through-hole in a center of the shaft body from one end to the other end;
a screw part provided on one end of the shaft body; and
a cutting portion for screwing is provided on the tip of the screw part.

2. A screw for fixing an atlantoaxial joint according to claim 1 wherein:
the cutting portion comprises a pair of acute protrusions facing each other formed by cutting tip of the screw in a state of inclination, and the protrusions comprise cutting blades.

3. A screw for fixing an atlantoaxial joint according to claim 1, wherein the shaft body comprises a tool-engaging hole.
